(19) United States
(12) Patent Application Publication
(10) Pub. No.: US 2020/0008665 A1
(43) Pub. Date: Jan. 9, 2020

(54) EXPANSION INSTRUMENT

(71) Applicant: Yoshihide TAKEKAWA, Komaki (JP)

(72) Inventor: Yoshihide TAKEKAWA, Komaki (JP)

(21) Appl. No.: 16/460,672

(22) Filed: Jul. 2, 2019

(30) Foreign Application Priority Data
Jul. 6, 2018 (JP) 2018-129060

(51) Int. Cl. A61B 1/32; A61B 1/303

(52) U.S. Cl. CPC A61B 1/32; A61B 1/31; A61B 1/303

(57) ABSTRACT

The expansion instrument includes an outer and inner circumference walls. The outer circumference wall is configured to be deformable in directions where a diameter of the outer circumference wall expands and shrinks. The inner circumference wall is located in a position surrounded by the outer circumference wall, and configured to be deformable in the directions where a diameter of the inner circumference wall expands and shrinks. Between the outer circumference wall and the inner circumference wall, an internal space where fluid can be introduced is provided. When the fluid is not introduced into the internal space, the outer and inner circumference walls are deformed in the direction where each diameter shrinks. When the amount of the fluid exceeding the initial volume that the internal space inherently has is introduced into the internal space, the outer and inner circumference walls are deformed in the direction where each diameter expands.

### CROSS-REFERENCE TO RELATED APPLICATION

This application claims the benefit of Japanese Patent Application No. 2018-129060 filed on Jul. 6, 2018 with the Japan Patent Office, the entire disclosure of which is herein incorporated by reference.

### BACKGROUND

The present disclosure relates to an expansion instrument.

Expansion instruments, which are inserted into a narrow space in a human body to expand the space, have been known. Examples of the expansion instruments may include vaginal speculums and anal speculums, which can be inserted into a body cavity and expand it. Japanese Unexamined Utility Model Application Publication No. S57-113102 suggests a vaginal speculum auxiliary membrane tube having a cylindrical membrane body formed of an elastic membrane to be used with a vaginal speculum.

In the conventional expansion instruments, a part of the instrument comes into contact with a part of the inner circumference of a body cavity in a circumferential direction. The contact area tends to be strongly pressed, which may cause feelings of discomfort and pain. The above-described vaginal speculum auxiliary membrane tube comes in contact with a whole inner circumference of the body cavity in the circumferential direction. However, since the cylindrical membrane body includes the conventional expansion instrument inside thereof, the contact area of the instrument tends to be strongly pressed. In this respect, the use of the vaginal speculum auxiliary membrane tube would make no difference from the case without the vaginal speculum auxiliary membrane tube.

### SUMMARY

In one aspect of the present disclosure, it is desirable to provide a new expansion instrument capable of expanding a whole inner circumference of a target expanding area.

One mode of the present disclosure is an expansion instrument comprising an outer circumference wall and an inner circumference wall. The outer circumference wall is configured to be deformable in a direction where a diameter of the outer circumference wall expands and in a direction where the diameter the outer circumference wall shrinks. The inner circumference wall is located in a position surrounded by the outer circumference wall and configured to be deformable in a direction where a diameter of the inner circumference wall expands and in a direction where the diameter of the inner circumference wall shrinks. Between the outer circumference wall and the inner circumference wall, an internal space where fluid can be introduced is provided. When an amount of the fluid not exceeding an initial volume that the internal space inherently has is introduced into the internal space, the outer circumference wall and the inner circumference wall may be deformed in the direction where each of the diameters shrinks. When the amount of the fluid exceeding the initial volume that the internal space inherently has is introduced into the internal space, the outer circumference wall and the inner circumference wall may be deformed in the direction where each of the diameters expands.

With the expansion instrument configured as such, the whole inner circumference of the target expanding area can be expanded. Thus, unlike the conventional expansion instrument, which presses a part of the inner circumference of the target expanding area, the expansion instrument reduces an excessive pressure on only a part of the inner circumference. For example, when the expansion instrument is configured to be inserted into the body cavity to expand the cavity, the expansion instrument comes in contact with the whole inner circumference of the body cavity, thereby causing less feelings of the discomfort and the pain by the contact point than the case of using the conventional product, which strongly and locally presses the contact point.

In one mode of the present disclosure, a plurality of partition walls may be provided between the outer circumference wall and the inner circumference wall, each of the plurality of partition walls radially extending from an outer circumferential side surface of the inner circumference wall to an inner circumferential side surface of the outer circumference wall, whereby the internal space may be divided to a plurality of divided spaces by the plurality of partition walls.

In one mode of the present disclosure, a through hole may be formed in the partition wall to communicate two divided spaces adjacent to each other across the partition wall.

In one mode of the present disclosure, the expansion instrument may be configured such that the expansion instrument is inserted into the body cavity in a state where the fluid is not introduced into the internal space, and then the fluid is introduced into the internal space, thereby expanding the body cavity.

### BRIEF DESCRIPTION OF THE DRAWINGS

FIG. 1A is a perspective view showing an expansion instrument having a shrunk diameter;

FIG. 1B is a perspective view showing the expansion instrument having an expanded diameter;

FIG. 2A is a sectional view showing a cross-section perpendicular to an axial direction of the expansion instrument having the shrunk diameter; and

FIG. 2B is a sectional view showing a cross-section perpendicular to the axial direction of the expansion instrument having the expanded diameter.

### DETAILED DESCRIPTION OF THE PREFERRED EMBODIMENTS

Hereinafter, the above-described expansion instrument will be described using exemplary embodiments.

### [Configuration of Expansion Instrument]

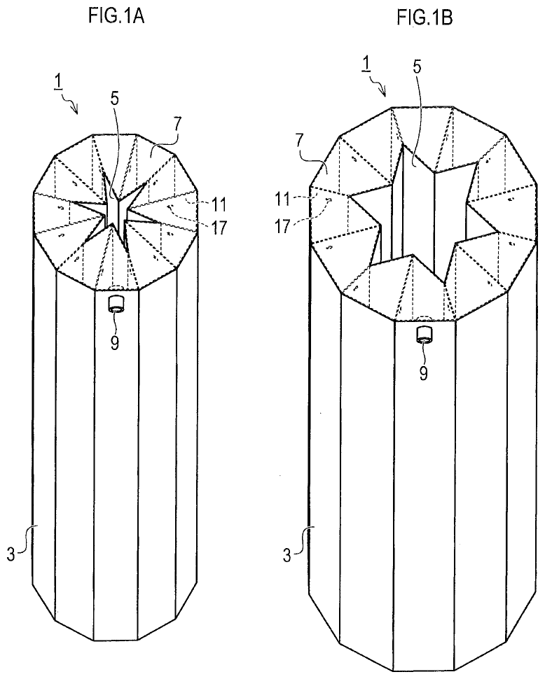

As shown in FIG. 1A and FIG. 1B, an expansion instrument 1 comprises an outer circumference wall 3, an inner circumference wall 5, an end wall 7 and an air plug 9. The outer circumference wall 3 is formed of soft materials, such as synthetic rubbers, which are flexibly deformable and extendable, and configured to be deformable in a direction where a diameter of the outer circumference wall 3 expands and in a direction where the diameter shrinks. The inner circumference wall 5 is also formed of the soft materials similar to the outer circumference wall 3, and configured to be deformable in a direction where a diameter of the inner circumference wall 5 expands and in a direction where the diameter shrinks. The end wall 7 is provided on both ends in an axial direction of the expansion instrument 1 (however, FIG. 1A and FIG. 1B illustrates only the end wall 7 provided on one end). The end wall 7 is also formed of the soft materials similar to the outer circumference wall 3.

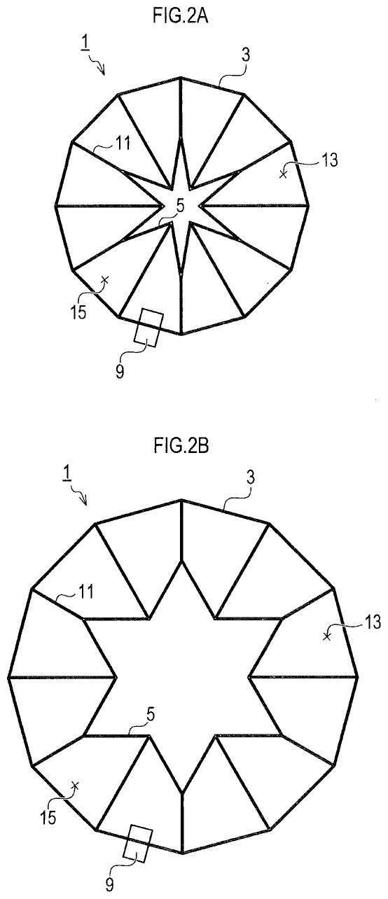

As shown in FIG. 2A and FIG. 2B, the expansion instrument 1 is provided with a plurality of partition walls 11 inside thereof (there are 12 partition walls 11 in this embodiment). The partition wall 11 is formed of a plastic plate with higher bending rigidity and flexibility than those of the outer circumference wall 3 and the inner circumference wall 5. The partition wall 11 is provided between the outer circumference wall 3 and the inner circumference wall 5 so as to extend radially from an outer circumferential side surface of the inner circumference wall 5 to an inner circumferential side surface of the outer circumference wall 3.

In a position surrounded by the outer circumference wall 3, the inner circumference wall 5 and both end walls 7, an internal space 13 into which fluid can be introduced is formed. The internal space 13 is divided into a plurality of divided spaces 15 by the plurality of partition walls 11 (there are 12 divided spaces 15 in this embodiment). Each of the plurality of partition wall 11 is provided with a through hole 17 formed therein as shown in FIG. 1A and FIG. 1B. Thus, two divided spaces 15 adjacent to each other across the partition wall 11 communicate via the through hole 17.

The outer circumference wall 3 is provided with an air plug 9 formed therein. The air plug 9 is configured to have a flow path, which can be opened and closed, for communicating an inner circumferential side and outer circumferential side of the outer circumference wall 3. Through this air plug 9, the air can be supplied to the internal space 13 from the outside. Also through this air plug 9, the air can be discharged from the internal space 13 to the outside. When the amount of the air not exceeding an initial volume that the internal space 13 inherently has is introduced into the internal space 13, the outer circumference wall 3 and the inner circumference wall 5 are deformed in the direction where each of the diameters shrinks as shown in FIG. 1A. As shown in FIG. 1A, since the outer circumference wall 3 and the inner circumference wall 5 are formed to have a polygonal cylindrical shape, the diameters may not be uniform unlike a diameter of a circular cylinder. However, even if there are a maximum diameter and a minimum diameter, the expansion instrument may shrink in the direction where each of the diameters shrinks. Therefore, both the “maximum diameter” and the “minimum diameter” are simply referred to as the “diameter” without distinctly differentiating them in this disclosure.

In a state where the diameters of the outer circumference wall 3 and the inner circumference wall 5 shrink, the air is supplied from the outside to the internal space 13 through the air plug 9. When the amount of the air exceeding the initial volume that the internal space 13 inherently has is supplied, the air flows into each of the plurality of divided space 15 via the through hole 17. Accordingly, the air is introduced into the whole of the internal space 13, and as a result, the outer circumference wall 3 and the inner circumference wall 5 are deformed to expand in the direction where each of the diameters expands as shown in FIG. 1B. As described above, even if there are the maximum diameter and the minimum diameter, the expansion instrument 1 expands in the direction where each of the diameters expands.

According to the expansion instrument 1 configured as described above, the inner circumference of the target expanding area can be expanded over the whole circumference. Thus, unlike the conventional expansion instrument that partially presses a part of the inner circumference of the target expanding area, the present expansion instrument 1 can reduce an excessive pressure on only a part of the inner circumference. For example, when the present expansion instrument 1 is configured to be inserted into the body cavity to expand it, the present expansion instrument 1 comes in contact with the whole inner circumference of the body cavity, thereby reducing the feelings of the discomfort and the pain occurred due to the contact point compared to the case of using the conventional product, which strongly presses a partial contact point.

The conventional expansion instrument has been formed of metals, such as stainless steel, and rigid plastic, for example. However, pressing the inside of the body cavity by a portion formed of such hard materials may often cause the feelings of discomfort and pain. In this regard, since the expansion instrument 1 is formed of soft materials that is flexibly deformable, the expansion instrument 1 can be deformed along the inside shape of the body cavity, thereby avoiding a concentration of pressure acting on the inside of the body cavity. Therefore, the use of such expansion instrument 1 as a vaginal speculum or an anal speculum can reduce the pressure in use and reduce the feelings of discomfort and pain.

In addition, since the expansion instrument 1 is provided with the plurality of partition walls 11, the expansion instrument 1 has moderate rigidity in an axial direction. Thus, when the expansion instrument 1 is inserted into a narrow place, it is possible to reduce a situation where the expansion instrument 1 is bent due to insertion resistance. Therefore, the expansion instrument 1 exhibits an excellent insertion property when inserted into the narrow place.

### Other Embodiments

The expansion instrument has been described using the exemplary embodiments; however, the above embodiments are merely examples of one mode of the present disclosure. That means the present disclosure is not limited to the above exemplary embodiments, and various modes of the present disclosure are applicable within a technical scope of the present disclosure.

For example, in the above-described embodiment, the exemplary materials forming the outer circumference wall 3, inner circumference wall 5 and end wall 7 include the soft materials, such as synthetic rubber, which are flexibly deformable and extendable. However, it may be possible to employ materials other than the synthetic rubber as the soft materials. Examples of the soft materials may include polyvinyl chloride, polyurethane, thermoplastic elastomers. Some of the soft materials may have less flexibility than the synthetic rubber, however, if dimensions of each part in an expanded state are configured to be similar to the dimensions used in the above embodiments, the outer circumference wall 3 and the inner circumference wall 5 can reduce the diameters by forming wrinkles and folds.

In the present disclosure, the diameters may shrink either by elasticity of the material itself, or by folding the folds of the material, which has low elasticity, so as to wrap around just like folding an umbrella. The combination thereof may also be employed. Alternatively, it may be possible to employ a structure in which the outer circumference wall 3 is formed of the synthetic rubber to impart the elasticity, and the inner circumference wall 5 is formed of the material with less elasticity than the synthetic rubber (polyvinyl chloride, for example) so that the outer circumference wall 3 shrinks by elasticity and the inner circumference wall 5 shrinks by folding the folds.

In the above embodiments, it is disclosed that the expansion instrument 1 may be used as the vaginal speculum and the anal speculum; however, the use of the expansion instrument of the present disclosure may not be limited to the insertion to the body cavity. Examples of other usage may include, for example, a case where an operator wishes to collect an object behind bushes of plants. In this case, the operator can insert the expansion instrument into the bushes of the plants, expand the space covered by the bushes, and collect the object without being disturbed by the bushes. At this time, it should be appreciated that appropriate dimensions may be employed for the diameter and axial length of the expansion instrument. If the operator collects the object by hands in the above situation, the diameter of the inner circumference wall 5 of the expansion instrument should have dimensions suitable for allowing the operator to insert his hands and arms, and the axial length of the expansion instrument should have dimensions suitable for the operator's arm length.

It may be also possible to configure a large expansion instrument so as to achieve a situation where the expansion instrument is vertically installed in the water and expanded, then an operator may enter the inside of the inner circumference wall 5 to do his work in a working space surrounded by the inner circumference wall 5. Since this configuration provides an environment where no floating objects or living things can enter the inside of the inner circumference wall 5, the operator can carry out his work without being disturbed by such floating objects and living things.

When the expansion instrument is installed in the water, the fluid used to expand the expansion instrument may be heavy fluid such as water, instead of the air. This reduces buoyancy acting on the expansion instrument. That means the fluid introduced into the expansion instrument of the present disclosure may not be limited to the air. If fluid heavier than water is needed, salt water and the like may be employed. When the expansion instrument is introduced into the body cavity, it is possible to reduce the feelings of discomfort caused by the expansion instrument touching the human body by introducing warm water, which has a substantially same temperature as a temperature of the body, into the expansion instrument.

What is claimed is:

1. An expansion instrument, comprising:
an outer circumference wall configured to be deformable in a direction where a diameter of the outer circumference wall expands and in a direction where the diameter of the outer circumference wall shrinks; and
an inner circumference wall located in a position surrounded by the outer circumference wall, the inner circumference wall being configured to be deformable in a direction where a diameter of the inner circumference wall expands and in a direction where the diameter of the inner circumference wall shrinks;
wherein between the outer circumference wall and the inner circumference wall, an internal space where fluid can be introduced is provided,
wherein when an amount of the fluid not exceeding an initial volume that the internal space inherently has is introduced into the internal space, the outer circumference wall and the inner circumference wall are deformed in the direction where each of the diameter of the outer circumference wall and the diameter of the inner circumference wall shrinks, and
wherein when an amount of the fluid exceeding the initial volume that the internal space inherently has is introduced into the internal space, the outer circumference wall and the inner circumference wall are deformed in the direction where each of the diameter of the outer circumference wall and the diameter of the inner circumference wall expands.

2. The expansion instrument according to claim 1,
wherein a plurality of partition walls is provided between the outer circumference wall and the inner circumference wall, each of the plurality of partition walls radially extending from an outer circumferential side of the inner circumference wall to an inner circumferential side of the outer circumference wall, whereby the internal space is divided into a plurality of divided spaces by the plurality of partition walls.

3. The expansion instrument according to claim 2,
wherein a through hole is formed in each of the plurality of partition walls, the through hole communicating two of the plurality of divided spaces adjacent to each other across each of the plurality of partition walls.

4. The expansion instrument according to claim 1,
wherein the expansion instrument is configured that the expansion instrument is inserted into a body cavity in a state where the amount of the fluid not exceeding the initial volume that the internal space inherently has is introduced into the internal space, and then the amount of the fluid exceeding the initial volume that the internal space inherently has is introduced into the internal space, thereby expanding the body cavity.
